(19) United States
(12) Patent Application Publication
(10) Pub. No.: US 2016/0231264 A1
(43) Pub. Date: Aug. 11, 2016

(54) Remotely Classifying Materials Based on Complex Permittivity Features

(71) Applicants: Nicholas C. Donnangelo, Purcellville, VA (US); Alex V. Mamishev, Seattle, WA (US); Adrian V. Mariano, Vienna, VA (US)

(72) Inventors: Nicholas C. Donnangelo, Purcellville, VA (US); Alex V. Mamishev, Seattle, WA (US); Adrian V. Mariano, Vienna, VA (US)

(73) Assignee: The Mitre Corporation, McLean, VA (US)

(21) Appl. No.: 12/395,250

(22) Filed: Feb. 27, 2009

(51) Int. Cl. G01R 27/00; G01N 33/22; G06N 5/02

(52) U.S. Cl. CPC G01N 27/02; G01N 33/22

(57) ABSTRACT

Provided are systems and methods for remotely classifying materials based on complex permittivity features. Such a system includes a first electrode, a second electrode, and a computing module. The first electrode is configured to generate an electric field. The second electrode is configured to sense interaction of the electric field with a container and any materials in the container and to provide a signal corresponding thereto. The computing module is configured to (i) convert the signal into one or more electrical parameters, (ii) classify the materials in the container based on the one or more electrical parameters, and (iii) identify at least one of the materials in the container to be contraband based on the classifications. The first and second electrodes may be configured as opposing parallel electrodes, a fringing-field sensor, or a combination thereof.

### STATEMENT REGARDING FEDERALLY SPONSORED RESEARCH AND DEVELOPMENT

Statement under MPEP 310. The U.S. government has a paid-up license in this invention and the right in limited circumstances to require the patent owner to license others on reasonable terms as provided for by the terms of 0706D070-DI, 0707D070-DI, and 0708D070-DI, awarded by the Defense Advanced Research Projects Agency (DARPA).

### BACKGROUND OF THE INVENTION

1. Field of the Invention

The present invention is generally directed to scanning items for concealed contraband, including but not limited to explosives, explosive precursors, and narcotics.

2. Background Art

Detection of contraband (such as, for example, explosives, explosive precursors, and narcotics) is a critical need of the U.S. Government (military, border control, and federal law enforcement), state and local law enforcement, and private security companies. Currently available systems for detecting contraband can be grouped into one of two categories: (i) residue-detection methods, which rely on physical residues or vapors to detect contraband; or (ii) nuclear-based methods, which use ionizing radiation to detect contraband.

Both the residue-detection methods and the nuclear-based methods have drawbacks. First, the residue-detection methods require access to a physical sample in order to detect contraband. Oftentimes, however, the contraband may be concealed, making residue detection difficult or impossible. For example, contraband that is odorless may be difficult to detect using residue-detection methods. Second, although nuclear-based methods do not require access to physical samples (like the residue-detection methods), the nuclear-based methods require ionizing (e.g., neutron) radiation, which can have deleterious effects on humans and/or the surrounding environment. Accordingly, nuclear-based methods are limited in their application by safety and cost considerations.

In addition to the residue-detection and nuclear-based methods, the food industry and the semiconductor industry use measurement methods based on the dielectric properties of materials to assess the quality of their respective products. Although the dielectric-based measurement methods do not have the same drawbacks as the residue-detection and nuclear-based methods, the dielectric-based measurement methods used by the food industry and the semiconductor industry are ill-suited for detecting contraband. Specifically, these dielectric-based measurement methods operate over very short ranges, have no imaging capability (e.g., are single voxel system), and are typically only used for sensing the presence of a single, targeted measurand (e.g., moisture content in cookies or purity of a pharmaceutical under manufacture).

For example, U.S. Pat. No. 7,280,940 to Goldfine et al., entitled “Segmented Field Dielectric Sensor Array for Material Characterization” (filed Mar. 7, 2006) (issued Oct. 9, 2007) describes representative measurement methods used for quality control in the semiconductor industry. Specifically, the '940 patent is “directed toward the nondestructive detection and characterization of insulating or semiconductor materials . . . ” '940 patent, col. 3 11. 31-33. According to the '940 patent, electrodes are placed in very close proximity with a material under test (“MUT”) to generate a two-dimensional grid used to estimate electrical properties of the MUT. Like the conventional measurement methods used by the semiconductor industry discussed above, the '940 patent teaches that the proximity, or “lift-off,” between the electrodes and the MUT is a very short range—on the order of a few millimeters to a few hundredths of a millimeter.

Given the foregoing, what is needed are methods, systems, and computer program products for remotely classifying materials based on complex permittivity features. The remote classification of materials could be used to identify contraband.

### BRIEF SUMMARY OF THE INVENTION

The present invention meets the above-described needs by providing methods, systems, and computer-program products for remotely classifying materials based on complex permittivity features. In accordance with embodiments of the present invention, the remotely classified materials are used to identify contraband.

For example, an embodiment of the present invention provides a system for identifying materials. The system includes a first electrode, a second electrode, and a computing module. The first electrode is configured to generate an electric field. The second electrode is configured to sense interaction of the electric field with a container and any materials in the container and to provide a signal corresponding thereto. The computing module is configured to (i) convert the signal into one or more electrical parameters, (ii) classify the materials in the container based on the one or more electrical parameters, and (iii) identify at least one of the materials in the container based on the classifications.

Another embodiment of the present invention provides a method for identifying materials. The method includes several steps. First, an electric field is generated. Second, interaction of the electric field with a container and any materials in the container is sensed to provide a signal. Third, the signal is converted into one or more electrical parameters. Fourth, the materials in the container are classified based on the one or more electrical parameters. Then, at least one of the materials in the container is identified based on the classifications.

A further embodiment of the present invention provides a tangible computer-readable medium having stored thereon computer-executable instructions that, if executed by a device, cause the device to perform a method for identifying materials. The method includes several steps. First, a signal, sensed by an electrode, is converted into one or more electrical parameters, wherein the electrode is configured to sense interaction of an electric field with a container and any materials in the container. Second, the materials in the container are classified based on the one or more electrical parameters. Then, at least one of the materials in the container is identified based on the classifications.

Further features and advantages of the invention, as well as the structure and operation of various embodiments of the invention, are described in detail below with reference to the accompanying drawings.

### BRIEF DESCRIPTION OF THE DRAWINGS/FIGURES

The accompanying drawings, which are incorporated herein and form part of the specification, illustrate the present invention and, together with the description, further serve to explain the principles of the invention and to enable a person skilled in the relevant art(s) to make and use the invention.

FIGS. 1A and 1B depict example systems for remotely classifying materials based on electrical parameters.

FIG. 2 depicts the real and imaginary components of the complex diectric permittivity of a material as a function of the frequency of an electric field used to excite the material.

FIGS. 3A and 3B depict example embodiments of systems for classifying materials included in an envelop.

FIGS. 4A-D depict example data obtained from the systems of FIGS. 3A and 3B.

FIG. 5 depicts example classification results obtained from the systems of FIGS. 3A and 3B.

FIG. 6 depicts another example embodiment of a system for classifying materials included in a sample.

FIGS. 7A and 7B depict example signatures for classifying materials included in a sample based on clustering of data points within a plot.

FIG. 8 depicts an example embodiment of a system for classifying materials included in an automobile.

FIGS. 9A and 9B depict example data obtained from the system of FIG. 8.

FIGS. 10A-D depict more example data obtained from the system of FIG. 8.

FIGS. 11A-D depict still more example data obtained from the system of FIG. 8.

FIGS. 12 and 13 depict example signatures for classifying materials included in a sample based on clustering of data points within a plot.

FIG. 14 depicts example classification results obtained from the system of FIG. 8.

FIG. 15 depicts example computer system that may be used in accordance with embodiments of the present invention.

The features and advantages of the present invention will become more apparent from the detailed description set forth below when taken in conjunction with the drawings, in which like reference characters identify corresponding elements throughout. In the drawings, like reference numbers generally indicate identical, functionally similar, and/or structurally similar elements. The drawing in which an element first appears is indicated by the leftmost digit(s) in the corresponding reference number.

### DETAILED DESCRIPTION OF THE INVENTION

### I. Overview

The present invention is directed to remotely classifying materials based on complex permittivity features of the materials. In this document, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

Embodiments of the present invention are directed to systems and methods for remotely detecting and classifying materials included in a container based on variations in electrical parameters (e.g., complex permittivity) of the materials. By remotely detecting and classifying materials, embodiments of the present invention may be used to detect contraband included in the container.

As used herein, the term “container” means a structure that holds or may be configured to hold goods, items, or materials. Example containers within the spirit and scope of the present invention may include, but are not limited to, an envelop, a jar, a box, a portable compartment (as may be used, for example, on a train, a ship, or a plane), a piece of luggage (such as, for example, a purse, a bag, a suitcase, a backpack, or the like), a vehicle (such as, for example, a train, a plane, or an automobile—including a car, a truck, a bus, and the like), or some other type of structure that holds or may be configured to hold goods, items, or materials.

As used herein, the term “contraband” means an illegal or prohibited good, item, or material. Examples of contraband may include, but are not limited to explosives, explosive precursors, narcotics, or some other type of illegal or prohibited good, item, or material.

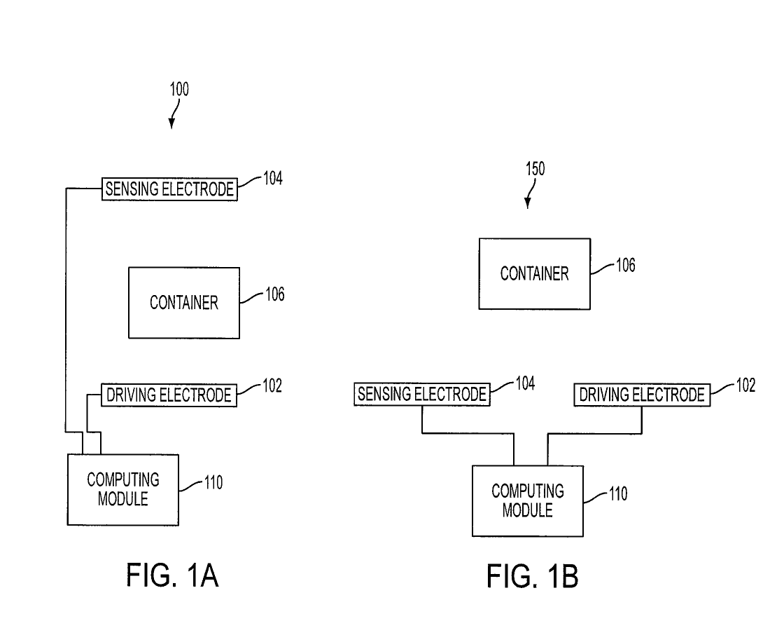

The classification of materials and detection of contraband in accordance with embodiments of the present invention is based on dispersive properties of a material positioned relative to a driving electrode and one or more sensing electrodes. The driving electrode and the one or more sensing electrodes may be configured as opposing plate electrodes (as illustrated, for example, in FIG. 1A), a fringing-field sensor array (as illustrated, for example, in FIG. 1B), or a combination thereof.

For example, FIG. 1A depicts an example system 100 in which electrodes are configured as opposing plate electrodes. System 100 includes a driving electrode 102 and a sensing electrode 104 configured as two opposing plate electrodes, with a container 106 located in between. Although system 100 is illustrated to include only one driving electrode 102 and one sensing electrode 104, it is to be appreciated that system 100 may include a plurality of driving electrodes and a plurality of sensing electrodes. In operation, driving electrode 102 is energized with a highly stable electric field. The electric field may have a single frequency (such as, for example, a sinusoidal waveform) or may have a plurality of frequencies (such as, for example, a substantially triangular waveform or a substantially square waveform). The electric field emanating from driving electrode 102 is distorted or modified by interstitial material included in container 106. The degree of distortion or modification of the electric field is dependent upon the interstitial material's dielectric characteristics, as well as other factors, as explained in more detail below. Sensing electrode 104 measures voltage potential caused by the electric field. Based on the voltage potential, a computing module 110—which is coupled to driving electrode 102 and sensing electrode 104—derives electrical parameters of the interstitial material. The electrical parameters may include, for example, impedance, phase, the (complex) dielectric constant, or other electrical parameters. Based on these electrical parameters, the computing module 110 classifies the material(s) included in container 106 and identifies whether container 106 includes contraband.

FIG. 1B depicts an example system 150 in which driving electrode 102 and sensing electrode 104 are configured as a fringing-field sensor array. In this system 150, driving electrode 102 and sensing electrode 104 are in a planar array with container 106 in a plane orthogonal to the plane of driving electrode 102 and sensing electrode 104. Like system 100, system 150 may include a plurality of driving electrodes 102 and a plurality of sensing electrodes 104. As mentioned above, the electric field may have a single frequency or may have a plurality of frequencies.

In operation, driving electrode 102 generates a fringing electric field that interacts with container 106 (and any materials therein) and is then sensed by sensing electrode 104. The distance between driving electrode 102 and sensing electrode 104 may be physically varied (e.g., increased) to vary (e.g., increase) the depth that the fringing electric field penetrates along the plane of container 106. Alternatively, when the system includes a plurality of driving electrodes and a plurality of sensing electrodes, the distance between the driving electrodes and the sensing electrodes may be effectively varied (e.g., increased) by varying the electrodes that are energized to generate the electric field and by varying the electrodes that are selected to sense the electric field. So, rather than mechanically moving electrodes, embodiments of the present invention electronically switch between electrodes to effectively adjust the distance between the electrodes used to generate and/or sense the electric field.

Like system 100 of FIG. 1A, system 150 includes computing module 110, which derives electrical parameters of interstitial material that may be included in container 106. In embodiments, both parallel-plate measurements of system 100 and fringing-field measurements of system 150 may be used to derive the electrical parameters of the interstitial materials, as the data produced by these two systems have different sensitivities.

The electrical parameter measurements provided by the parallel-plate configuration of system 100 and/or the fringing-field configuration of system 150 are processed by computing module 110. Computing module 110 may implement any of a variety of classification algorithms. Multiple electrode combinations may be used to produce a map of dielectric properties and classification within different volume elements. The presence of sharp discontinuities in dielectric properties is a macro-indication that the test article may warrant further investigation, and classification based on known signatures for contraband substances may be conclusive.

In embodiments, for example, computing module 110 may implement linear discriminant analysis (LDA) to develop signatures for identifying contraband substances based on the electrical parameters. As explained in more detail below, LDA is a method for separating the electrical parameters into different clusters corresponding to the different types of materials that may be included in container 106.

As mentioned above, the degree to which a material in container 106 distorts or modifies an applied electric field is dependent on dispersive properties of the material. The dispersive properties of a material can be understood, for example, in terms of the complex permittivity, which comprises a dielectric constant (relating the applied electric field to a displacement field within the material) and a frequency-dependent conductivity (relating the applied electric field to a current density within the material). The complex permittivity of a material can be represented mathematically as follows:

∈*=∈′+i∈″

wherein ∈* is the complex permittivity, ∈′ is the dielectric constant or the real part of the complex permittivity, and ∈″ is the imaginary part of the complex permittivity. The imaginary part of the complex permittivity can be represented mathematically as follows:

$${ɛ}^{″} = \frac{σ}{2 π f}$$

wherein σ is the conductivity of the material and f is the frequency of the applied electric field.

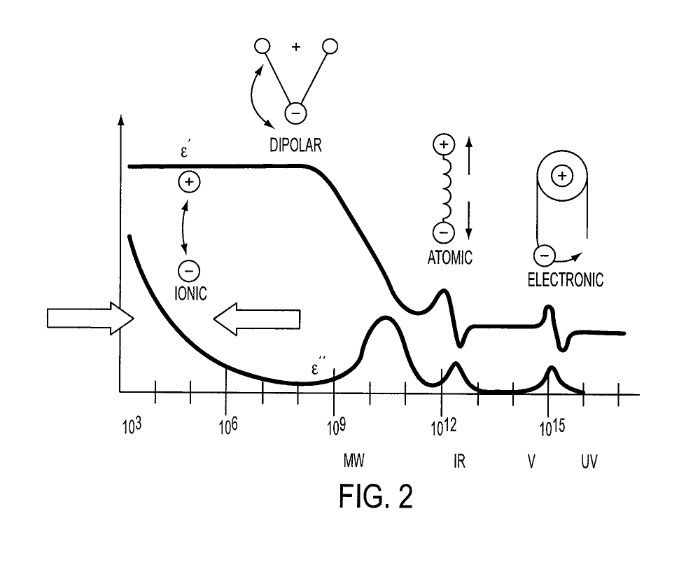

FIG. 2 depicts a schematic plot of the real part (∈′) and the imaginary part (∈″) of the complex permittivity of a material as a function of the frequency of the applied electric field. At optical frequencies (or wavelengths), molecular vibration and rotation of molecules and inter atomic bonds lead to the detailed structure observed in infrared spectroscopy. At extremely low excitation frequencies (e.g., 1 Hz to 500,000 Hz), polarized dipoles re-align to neutralize the effect of the applied field. This re-alignment of dipoles occurs to a varying extent for different materials and gives rise to changes in the complex permittivity of materials. These changes over a span of frequencies form a signature that can be exploited for material identification. The significant dispersion of dielectric properties of materials occurs at both the extremely low end of the spectra and in the millimeter wave and higher region. Some embodiments of the present invention operate at extremely low frequencies (ELF), while other embodiments operate in the millimeter and Terahertz range.

For illustrative purposes, and not limitation, three embodiments are described below in which the container respectively comprises an envelop, a jar, and an automobile. It is to be appreciated, however, that other forms of containers may be used without deviating from the spirit and scope of the present invention.

### II. Example Embodiment for Classifying Materials Included in an Envelop

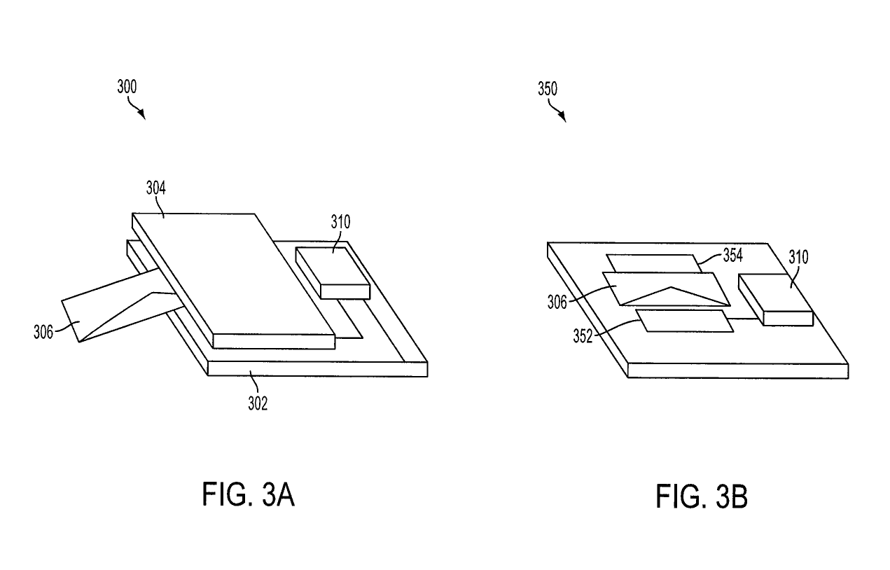

FIGS. 3A and 3B respectively illustrate an example parallel-plate system 300 and an example fringing-field system 350 for classifying materials included in an envelop 306. Using system 300, system 350, or a combination thereof, contraband included within envelop 306 may be detected.

Referring to FIG. 3A, system 300 includes a driving electrode 302, a sensing electrode 304, and a computing module 310 coupled to each electrode. Driving electrode 302 and sensing electrode 304 are arranged as opposing plate electrodes. In an embodiment, the separation between driving electrode 302 and sensing electrode 304 is approximately 15 millimeters, and the measurement surface is approximately 6.5 inches by approximately 9.74 inches.

Referring to FIG. 3B, system 350 includes a driving electrode 352, a sensing electrode 354, and computing module 310 coupled to each electrode. In system 350 driving electrode 352 and sensing electrode 354 are arranged as a fringing-field array.

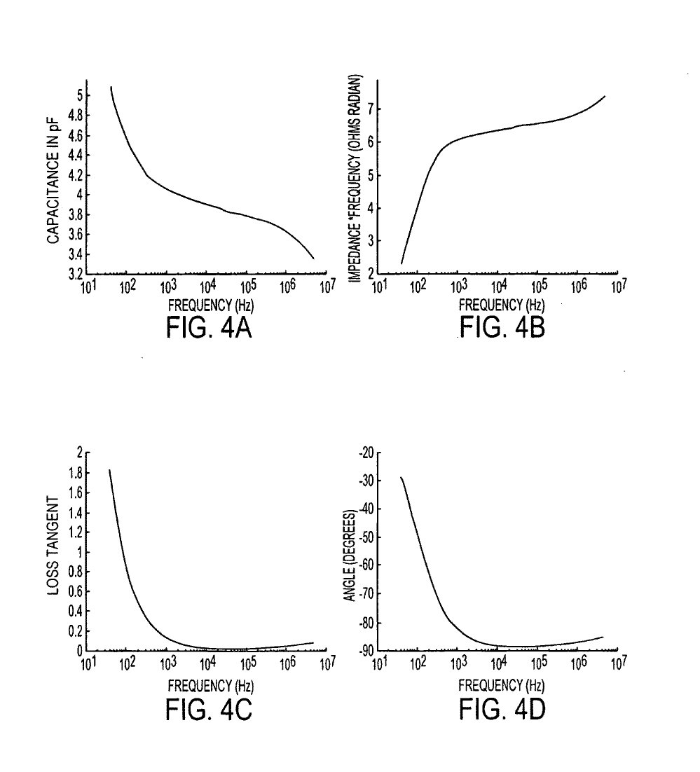

In both system 300 and system 350, driving electrodes 302, 352 respectively generate an electric field that interacts with envelop 306 and any materials included therein. The interaction of the electric field with envelop 306 and any materials therein distorts the electric field. Sensing electrodes 304, 354 sense the distorted electric field to provide a signal. Computing module 310 receives the signal and derives electrical parameters of the materials included in envelop 306. For example, computing module 310 may derive data as illustrated in FIGS. 4A-4D.

Based on such data, computing module 310 classifies materials included in envelop 306. In embodiments, computing module 310 classifies the materials based on linear discriminant analysis (LDA), which is a mathematical technique for identifying a subspace in which data has the largest variance. In this way, the data of the electrical parameters of the materials can potentially be organized in clusters, wherein each cluster of data corresponds to a different material included in envelop 306. LDA is described in more detail below.

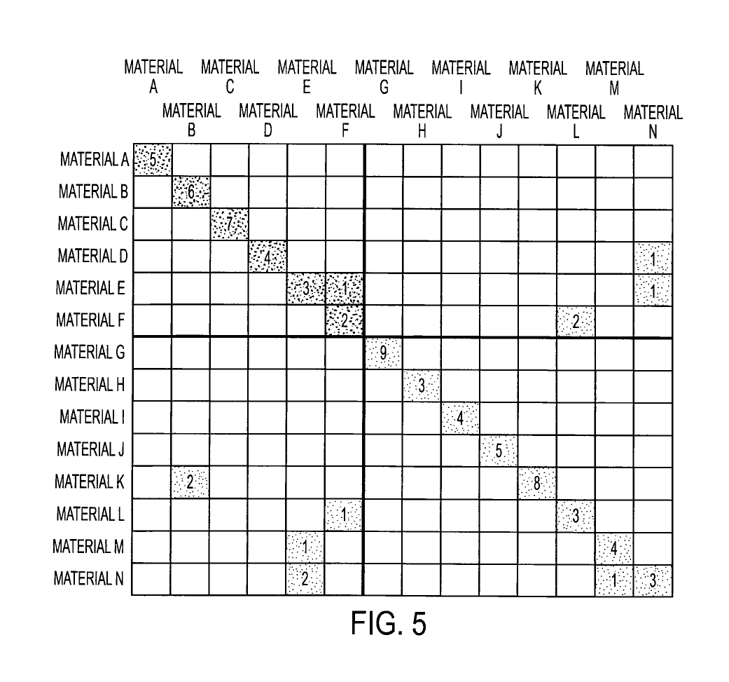

FIG. 5 illustrates example results from an experiment to classify materials using a system like the ones shown in FIGS. 3A and 3B. In FIG. 5, the materials listed on the rows represent materials that were included in envelop 306. The materials listed on the columns represents the classifications made by computing module 310. So, for example, computing module 310 correctly identified air nine times; in contrast, computing module 310 correctly identified potassium chlorate only three times and incorrectly identified potassium chlorate two times (once as potassium nitrate and once as sodium bicarbonate).

### III. Example Embodiment for Classifying Materials Included in a Jar

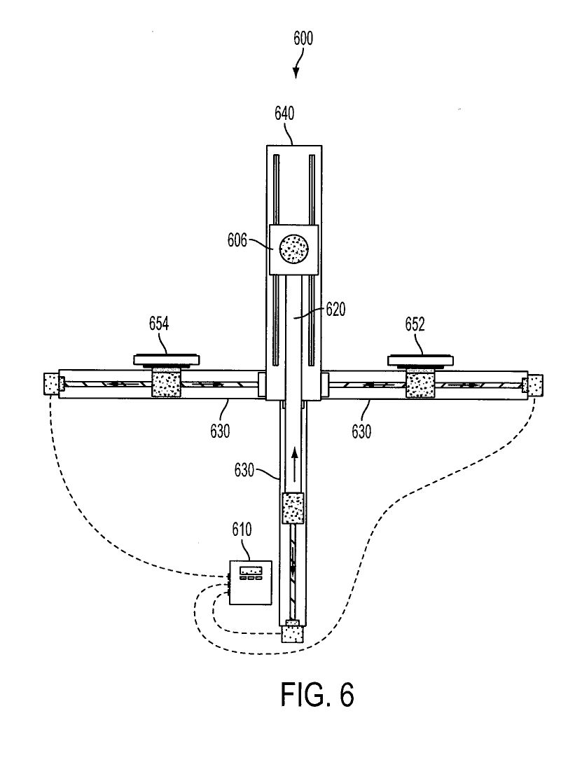

FIG. 6 illustrates an example system 600 for classifying materials included in a jar. Referring to FIG. 6, system 600 includes a driving electrode 652, a sensing electrode 654, a motor driver 610, and a platform 606. Driving electrode 652 and sensing electrode 654 are positioned along a first (e.g., transverse) arm of aluminum segment 630. Platform 606 is configured to hold a jar (or other test object) and is coupled to a non-conductive linkage 620. Non-conductive linkage is configured to move along a second (e.g., longitudinal) arm of aluminum segment 630 and a non-conductive segment 640 that is in a line with the second arm of aluminum segment 630. Motor driver 610 is configured to: (1) cause driving electrode 652 and sensing electrode 654 to move along the first (e.g., transverse) segment of aluminum segment 630, thereby adjusting the distance between driving electrode 652 and sensing electrode 654; and/or (2) cause non-conductive linkage 620 to move along the second (e.g., longitudinal) arm of aluminum segment 630, thereby adjusting the distance between a sample positioned on platform 606 and driving electrode 652 and sensing electrode 654. Motor driver 610 is coupled to a computing module (not shown), which is configured to send commands to motor driver 610 and to process data from sensing electrode 654.

The computing module may implement one or more methods for classifying materials included in a sample positioned on platform 606. For example, the computing module may implement a Bayesian-classification method. The computing module may then compute, for example, the Bhattacharyya distance between materials included in the sample. In general, the Bhattacharyya distance measures the similarity of two discrete probability distributions. In this context, the Bhattacharyya distance may be used as a measure for assessing the performance of the Bayesian-classification method. Table 1 includes the Bhattacharyya distance for a classifying various materials included in a sample. Assuming equal prior probabilities for two probability distributions, the Bhattacharyya distance, B, bounds the Bayes error (i.e., error<exp(−B)). This means that B>10 gives an error rate lower than 2e-5, B>5 gives an error rate lower than 0.3%, and B>2 gives an error rate lower than 6.8%.

TABLE 1

| | Material A | Material B | Material C | Material D | Material E | Material F | Material G | Material H | Material I | Material J |
|---|---|---|---|---|---|---|---|---|---|---|
| Material A | 0 | 607 | 2342 | 3.89 | 442 | 3067 | 3668 | 4042 | 1603 | 1076 |
| Material B | 607 | 0 | 610 | 563 | 482 | 1050 | 1325 | 1664 | 345 | 234 |
| Material C | 2342 | 610 | 0 | 2273 | 1821 | 501 | 476 | 818 | 465 | 760 |
| Material D | 3.89 | 563 | 2273 | 0 | 451 | 2984 | 3551 | 3951 | 1551 | 1041 |
| Material E | 442 | 482 | 1821 | 451 | 0 | 1753 | 2471 | 2416 | 715 | 332 |
| Material F | 3067 | 1050 | 501 | 2984 | 1753 | 0 | 114 | 71 | 243 | 570 |
| Material G | 3668 | 1325 | 476 | 3551 | 2471 | 114 | 0 | 147 | 538 | 994 |
| Material H | 4042 | 1664 | 818 | 3951 | 2416 | 71 | 147 | 0 | 558 | 996 |
| Material I | 1603 | 345 | 465 | 1551 | 715 | 243 | 538 | 558 | 0 | 74 |
| Material J | 1076 | 234 | 760 | 1041 | 332 | 570 | 994 | 996 | 74 | 0 |

System 600 can be used to collect various data used to classify materials included in a container positioned on platform 606. In operation, driving electrode 652 generates an electric field. Sensing electrode 654 senses distortions in the electric field based on the interaction of the electric field with the container (and materials therein) positioned on platform 606. Data regarding the distortion of the electric field is collected by a computing module (not shown). Data may be collected for various frequencies of the electric field generated by driving electrode 652, for various distances between driving electrode 652 and sensing electrode 654, and/or for various distances between platform 606 and the first (e.g., transverse) arm of segment 630.

From the data of the distortion of the electric field, the computing module can derive electrical parameters of the materials included in the container. For example, the computing module may derive the complex permittivity of the materials as a function of frequency. In embodiments, the data of the electrical parameters is organized into multi-dimensional vectors. The multi-dimensional vectors are projected into a lower-dimensional subspace using LDA. Importantly, the data may be grouped into distinct clusters in the lower-dimensional subspace, wherein the distinct clusters represent distinct materials included in the container. In this way, each cluster may serve as a signature for classifying the materials included in the container.

To further illustrate how computing module may classify materials, example data collected from system 600 is presented below. It is to be appreciated, however, that this example data is presented for illustrative purposes only, and not limitation. In this example data, five classes of materials were tested: air, salt, sugar, starch, and flour. The data was taken from a fixed distance (i.e., the separation between the materials and the first (e.g., transverse) arm of segment 630 was fixed). Twenty samples of air, salt, and starch and seventy samples of sugar and flour were used. The computing module derived the complex impedance of the samples taken at six different frequencies, resulting in 12-dimensional sample vectors (6 real components and 6 imaginary components). The mean air signature was subtracted from all the data. The 12-dimensional sample vectors were projected into a two-dimensional subspace using Fisher LDA.

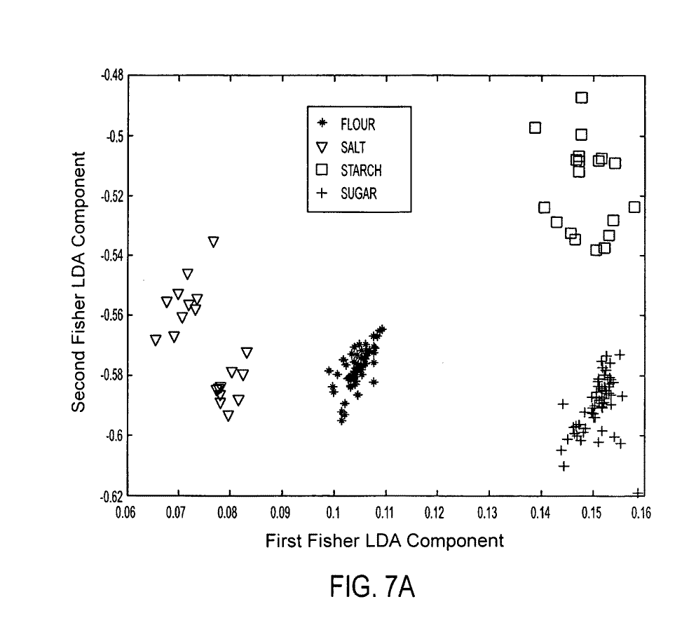

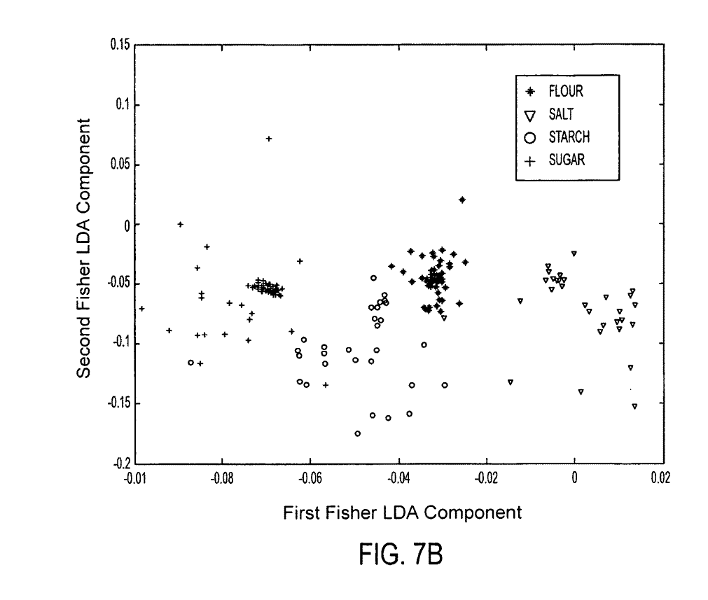

FIGS. 7A and 7B illustrate example plots of the data when projected into the two-dimensional subspace using Fisher LDA. In particular, FIG. 7A illustrates the clustering of the data when the distance between the samples and the electrodes remains fixed; whereas FIG. 7B illustrates the clustering of the data when the distance between the samples and the electrodes varies. As illustrated in FIG. 7A, the data falls into four distinct clusters corresponding to the four classes of materials (other than air) included in the container: salt, sugar, starch, and flour. Accordingly, the clustering serves as a signature for classifying materials.

The Bhattacharyya distance for classifying various materials using system 600 of FIG. 6 are illustrated below in Table 2 and Table 3. As mentioned above, the Bhattacharyya distance measures the similarity of two discrete probability distributions. Table 2 illustrates the Bhattacharyya distances when the distance between the samples and the electrodes was in a first range of distances, and Table 3 illustrates the Bhattacharyya distances when the distance between the samples and the electrodes was in a second range of distances.

TABLE 2
Bhattacharyya distances for 16 cm to 26 cm range

| | Starch | Flour | Salt | Sugar | Water |
|---|---|---|---|---|---|
| Starch | 0 | 3.1 | 1.7 | 1.0 | 2.4 |
| Flour | 3.1 | 0 | 3.2 | 5.0 | 7.1 |
| Salt | 1.7 | 3.2 | 0 | 4.3 | 6.8 |
| Sugar | 1.0 | 5.0 | 4.3 | 0 | 1.7 |
| Water | 2.4 | 7.1 | 6.8 | 1.7 | 0 |

TABLE 3
Bhattacharyya distances for 50 cm to 80 cm range

| | Starch | Flour | Salt | Sugar | Water |
|---|---|---|---|---|---|
| Starch | 0 | 0.66 | 0.49 | 0.52 | 3.8 |
| Flour | 0.66 | 0 | 0.46 | 0.36 | 3.5 |
| Salt | 0.49 | 0.46 | 0 | 0.53 | 4.0 |
| Sugar | 0.52 | 0.36 | 0.53 | 0 | 3.8 |
| Water | 3.8 | 3.5 | 4.0 | 3.8 | 0 |

### IV. Example Embodiment for Classifying Materials Included in an Automobile

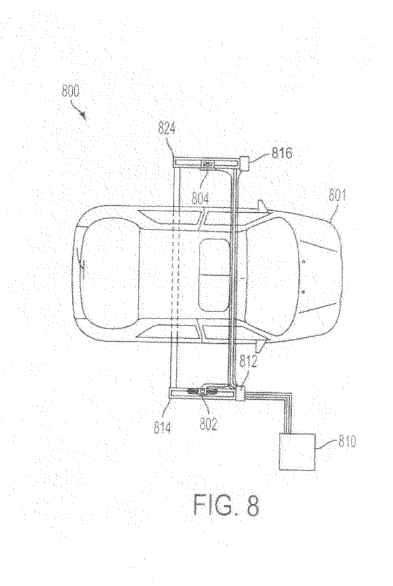

FIG. 8 depicts an example system 800 for classifying materials included in an automobile 801. System 800 includes a driving electrode 802 mounted on a linear guide 814 and a sensing electrode 804 mounted on a linear guide 824. Driving electrode 802 and sensing electrode 804 are coupled to a computing module 810, which sends instructions to and receives data from driving electrode 802 and/or sensing electrode 824. Computing module 810 is also coupled to motors 812 and 816. Motor 812 is configured to cause driving electrode 802 to move along linear guide 814, and motor 816 is configured to cause sensing electrode 804 to move along linear guide 824.

System 800 may be used, for example, to detect contraband (e.g., explosives, explosive precursors, and/or narcotics) included in automobile 801. In operation, driving electrode 802 generates an electric field, and sensing electrode 804 senses distortions in the electric field after the electric field interacts with materials in the automobile 801, in a similar manner to the embodiments described above. And, like the embodiments discussed above, computing module 810 classifies the materials included in automobile 801 based on electrical parameters of the materials derived from the distortions of the electric field.

To further illustrate how system 800 may be used to detect the presence of contraband, example data collected from system 800 is presented below. It is to be appreciated, however, that this example data is presented for illustrative purposes only, and not limitation. In this example, several different types of explosive materials and several different types of relatively benign materials were tested. Data were taken over a fairly broad frequency range from approximately 10 Hz to 40 kHz. The higher frequencies were measured first. The spacing between driving electrode 802 and sensing electrode 804 was fixed. Each sample vector is 12 dimensional (including 6 real components and 6 imaginary components).

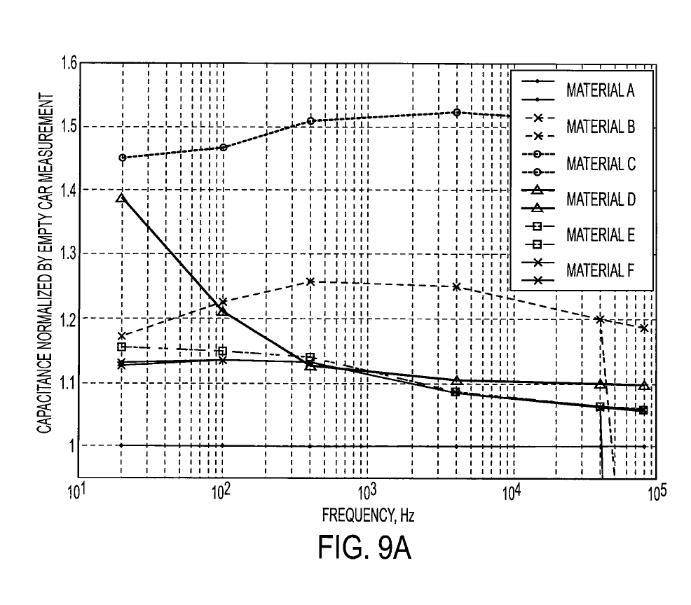

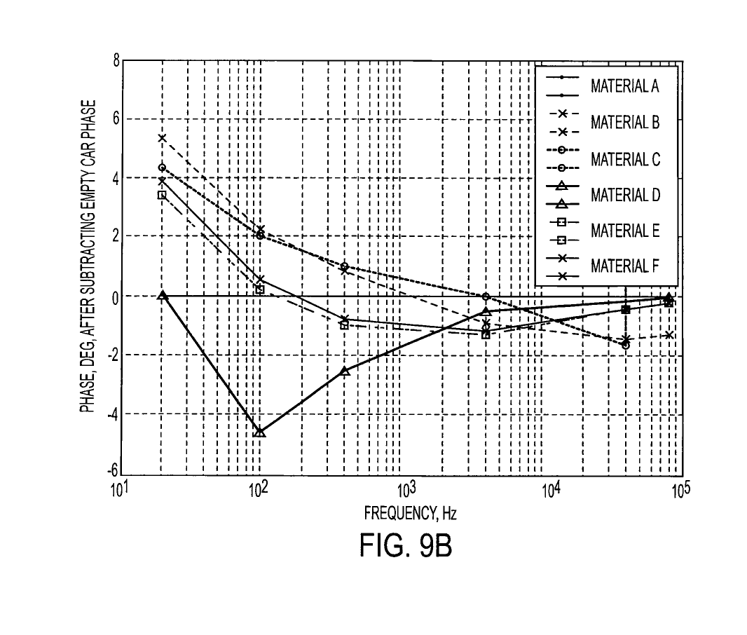

FIGS. 9A and 9B illustrate example pre-measurement signatures obtained by system 800. Specifically, FIG. 9A illustrates pre-measured signatures of the capacitance of the various materials as a function of the frequency of the applied electric field, and FIG. 9B illustrates pre-measured signatures of the phase of the various materials as a function of the frequency of the applied electric field.

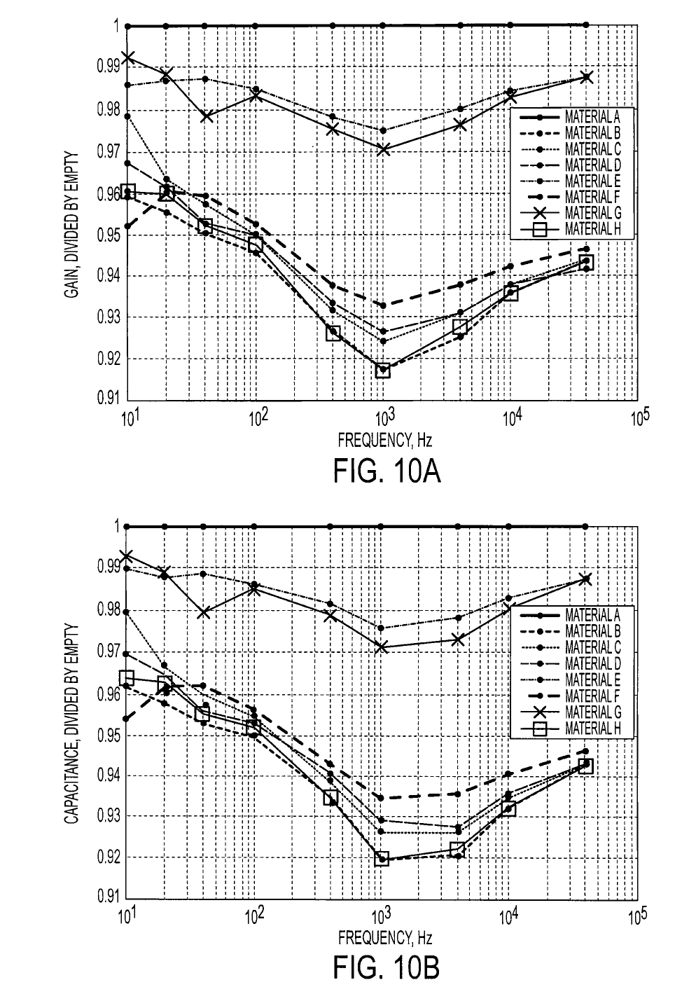

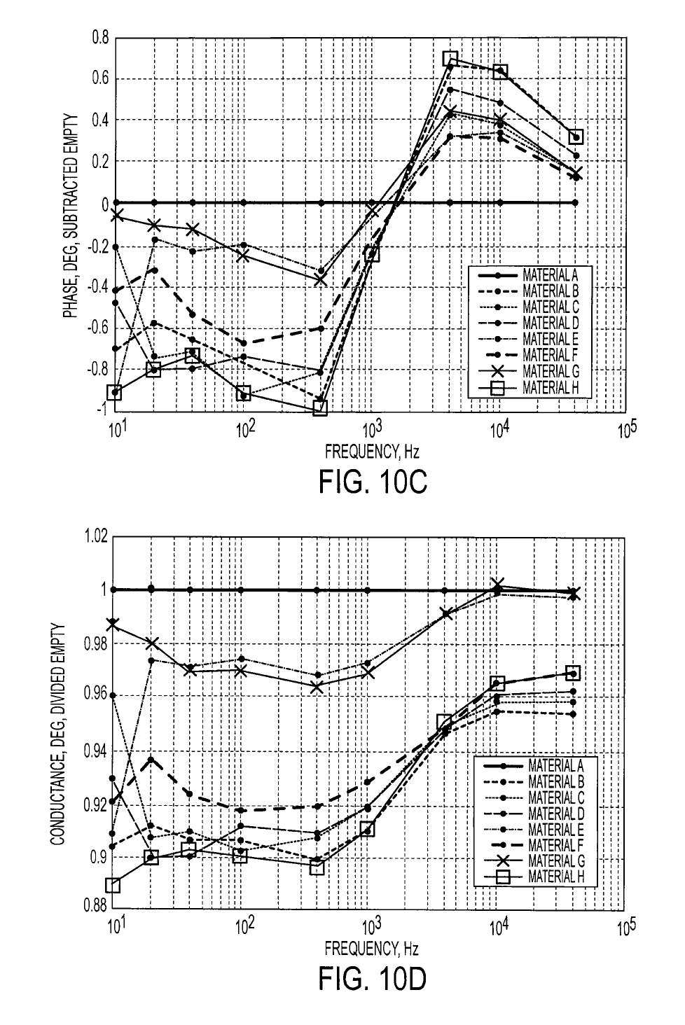

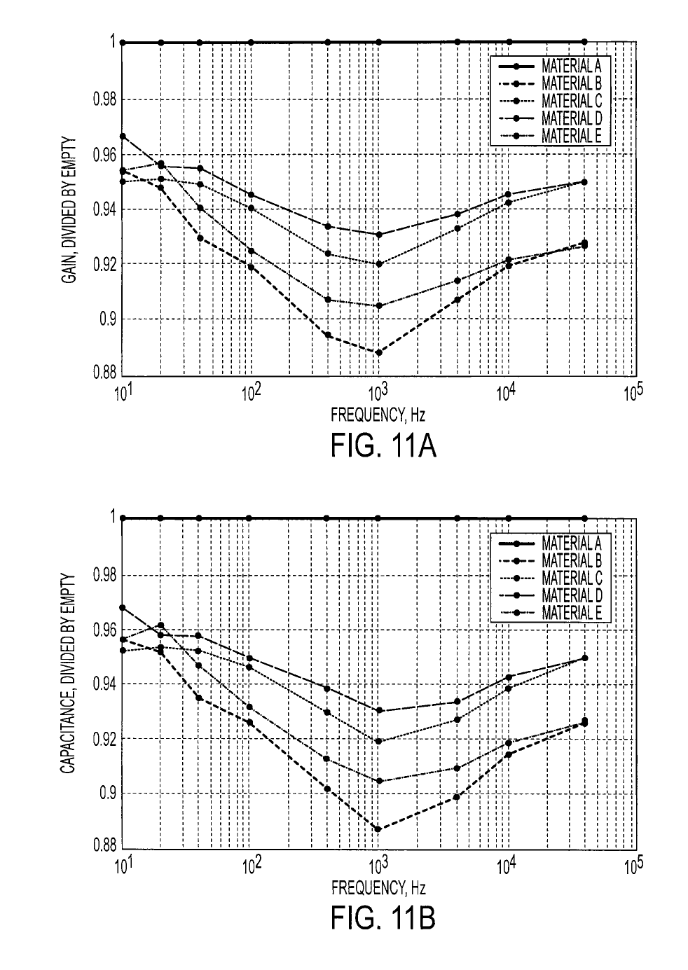

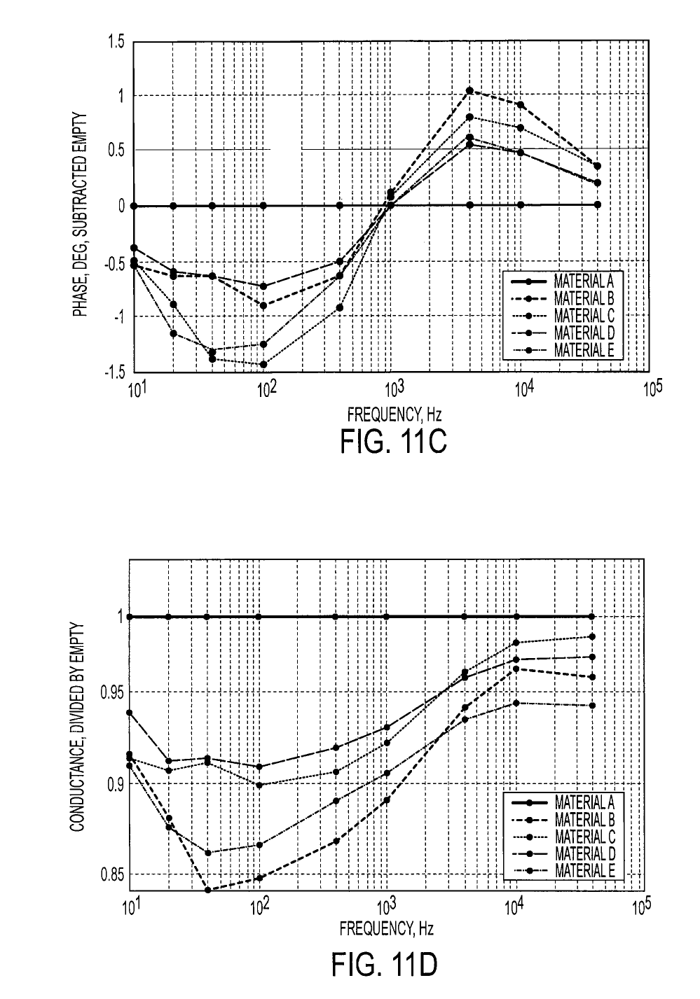

In addition to the pre-measurement signatures, system 800 may be used to obtain data used to derive electrical parameters of materials included in automobile 801. For example, FIGS. 10A-D illustrate plots of electrical parameters of the various materials as a function of the frequency of the applied electric field when the electrodes are at a first fixed position. Similarly, FIGS. 11A-D illustrates plots of electrical parameters of the various materials as a function of the frequency of the applied electric field when the electrodes are at a second fixed position. Specifically, FIGS. 10A and 11A depict plots of the gain of the electric field as a function of frequency; FIGS. 10B and 11B depict plots of the capacitance of the various materials as a function of frequency; FIGS. 10C and 11C depict plots of the phase of the electric field as a function of frequency; and FIGS. 10D and 11D depict plots of the conductance of the various materials as a function of frequency.

Computing module 810 may implement one or more methods to classify the materials in automobile 801. For example, according to a first example method, computing module 810 compares normalized blind measurements with the normalized pre-measured signatures illustrated in FIGS. 9A and 9B. According to this method, computing module 810 calculates the deviation between the normalized blind measurements of capacitance and the pre-measurement signatures of capacitance (which are illustrated in FIG. 9A). Based on this method, the pre-measured sample with the smallest absolute value deviation to the unknown sample is identified as a match.

According to a second example method, computing module 810 computes a Bayesian-classification method. The performance of the Bayesian-classification method can be assessed using the Bhattacharyya distance. As mentioned above, the Bhattacharyya distance measures the similarity of two discrete probability distributions and may be used to measure the separability of classes in a classification. The Bhattacharyya distances for the above-mentioned example materials are presented below in Table 4. Assuming equal prior probabilities for two probability distributions, the Bhattacharyya distance, B, bounds the Bayes error (i.e., error<exp(−B)). This means that B>10 gives an error rate lower than 2e-5, B>5 gives an error rate lower than 0.3%, and B>2 gives an error rate lower than 6.8%.

TABLE 4
Bhattacharyya distances for example materials tested
using the system of FIG. 8

| | Material A | Material B | Material C | Material D | Material E | Material F | Material G | Material H |
|---|---|---|---|---|---|---|---|---|
| Material A | 0 | 35.63 | 277.70 | 32.60 | 5.11 | 35.57 | 37.52 | 221.69 |
| Material B | 35.63 | 0 | 120.80 | 1.72 | 48.60 | 1.59 | 1.67 | 84.03 |
| Material C | 277.70 | 120.80 | 0 | 125.50 | 309.93 | 118.38 | 116.59 | 5.04 |
| Material D | 32.60 | 1.72 | 125.50 | 0 | 46.93 | 1.27 | 0.59 | 87.80 |
| Material E | 5.11 | 48.60 | 309.93 | 46.93 | 0 | 49.39 | 52.66 | 252.50 |
| Material F | 35.57 | 1.59 | 118.38 | 1.27 | 49.39 | 0 | 1.10 | 82.51 |
| Material G | 37.52 | 1.67 | 116.59 | 0.59 | 52.66 | 1.10 | 0 | 80.22 |
| Material H | 221.69 | 84.03 | 5.04 | 87.80 | 252.50 | 82.51 | 80.22 | 0 |

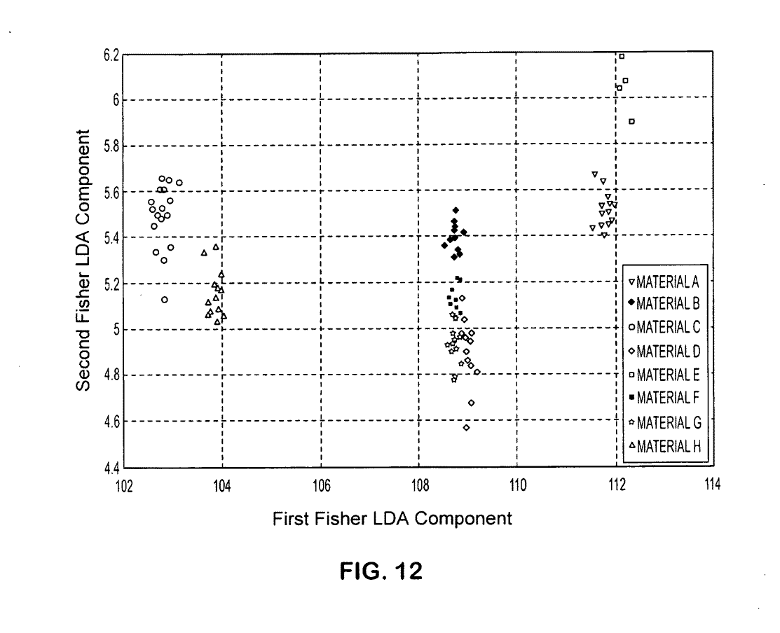

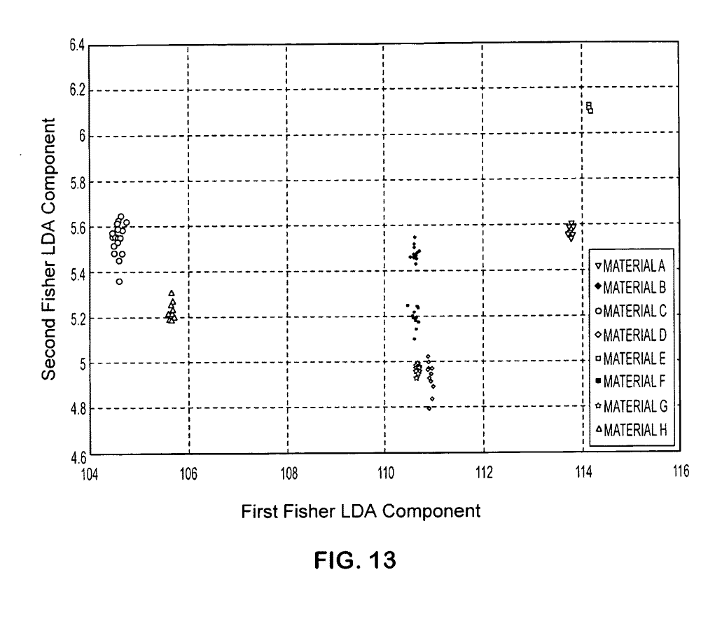

According to a third example method, computing module 810 implements LDA to find a linear combination of features that best separates the classes of materials included in automobile 801. For example, FIG. 12 illustrates the results of LDA implemented on the above-mentioned materials, wherein the horizontal and vertical dimensions in the plot of FIG. 12 represent the best cross-section of a 7-dimensional space. FIG. 13 illustrates the horizontal and vertical dimensions of FIG. 12 proportionally mapped to a third dimension to show an equivalent cluster density (which is not to be confused with separability).

According to a fourth example method, computing module 810 implements a partial least squares fit. Unlike a conventional least squares fit, a partial least squares fit is well-suited for blind tests, but requires extensive preliminary measurements prior to material identification. According to a partial least squares fit, prediction functions are extracted from cross-product matrices involving both a response variable, Y, and an independent variable, X. Compared to a conventional least squares fit, calibrations in a partial least squares fit are generally more robust, provided that the calibration set accurately reflects the range of variability expected in unknown samples.

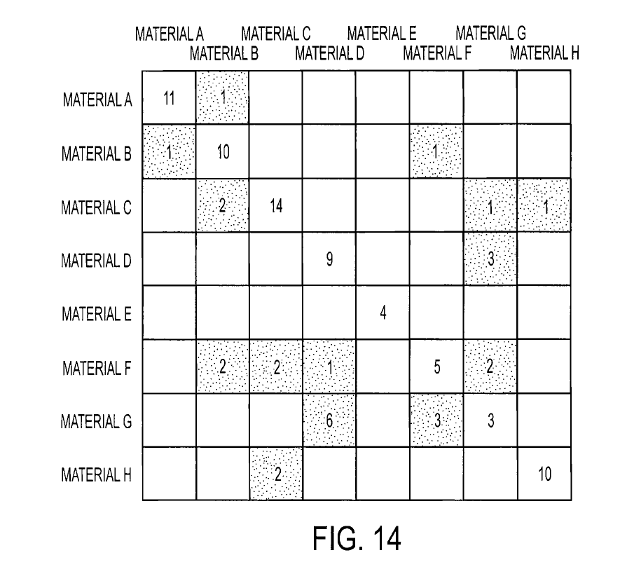

FIG. 14 illustrates example results obtained from a partial least squares fit. It is to be appreciated that these results are intended for illustrative purposes only, and not limitation. In FIG. 14, the rows show what the true measurements are, and the columns show what computing module 810 concluded the material is.

### V. Example Computer System

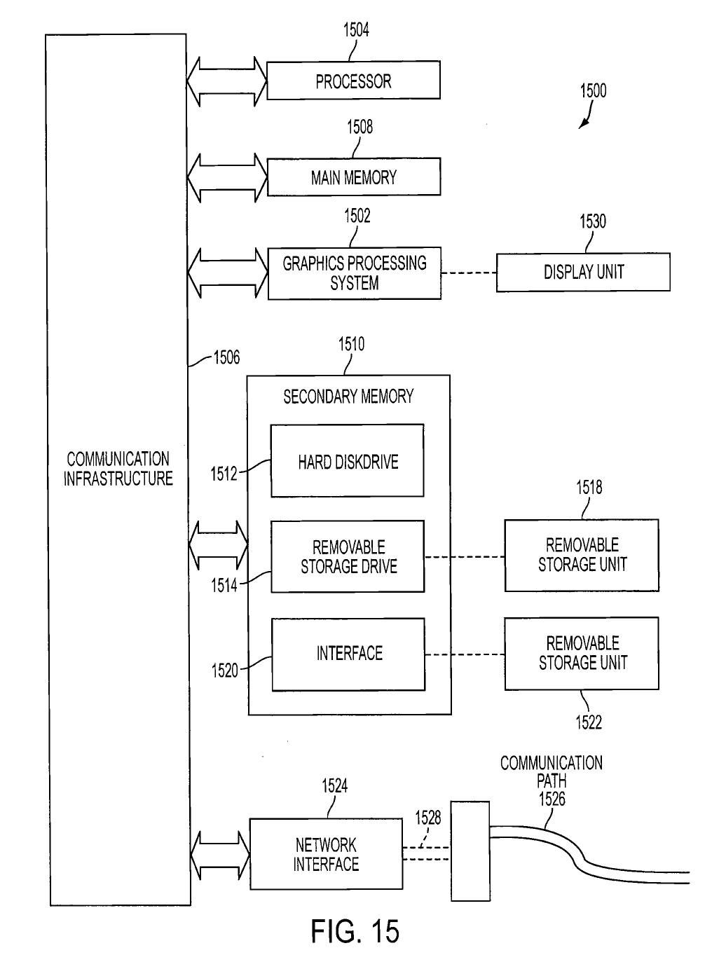

Various aspects of the present invention—such as the computing modules described herein—can be implemented by software, firmware, hardware, or a combination thereof. FIG. 15 illustrates an example computer system 1500 in which an embodiment of the present invention, or portions thereof, can be implemented as computer-readable code. Various embodiments of the invention are described in terms of this example computer system 1500. After reading this description, it will become apparent to a person skilled in the relevant art how to implement the invention using other computer systems and/or computer architectures.

Computer system 1500 includes one or more processors, such as processor 1504. Processor 1504 can be a special purpose or a general purpose processor. Processor 1504 is connected to a communication infrastructure 1506 (for example, a bus or network). Computer system 1500 may also include a graphics processing system 1502 for rendering images to an associated display 1530.

Computer system 1500 also includes a main memory 1508, preferably random access memory (RAM), and may also include a secondary memory 1510. Secondary memory 1510 may include, for example, a hard disk drive 1512 and/or a removable storage drive 1514. Removable storage drive 1514 may comprise a floppy disk drive, a magnetic tape drive, an optical disk drive, a flash memory, or the like. The removable storage drive 1514 reads from and/or writes to a removable storage unit 1518 in a well known manner. Removable storage unit 1518 may comprise a floppy disk, magnetic tape, optical disk, etc. which is read by and written to by removable storage drive 1514. As will be appreciated by persons skilled in the relevant art(s), removable storage unit 1518 includes a computer usable storage medium having stored therein computer software and/or data.

In alternative implementations, secondary memory 1510 may include other similar means for allowing computer programs or other instructions to be loaded into computer system 1500. Such means may include, for example, a removable storage unit 1522 and an interface 1520. Examples of such means may include a program cartridge and cartridge interface (such as that found in video game devices), a removable memory chip (such as an EPROM, or PROM) and associated socket, and other removable storage units 1522 and interfaces 1520 which allow software and data to be transferred from the removable storage unit 1522 to computer system 1500.

Computer system 1500 may also include a communications interface 1524. Communications interface 1524 allows software and data to be transferred between computer system 1500 and external devices. Communications interface 1524 may include a modem, a network interface (such as an Ethernet card), a communications port, a PCMCIA slot and card, or the like. Software and data transferred via communications interface 1524 are in the form of signals 1528 which may be electronic, electromagnetic, optical, or other signals capable of being received by communications interface 1524. These signals 1528 are provided to communications interface 1524 via a communications path 1526. Communications path 1526 carries signals 1528 and may be implemented using wire or cable, fiber optics, a phone line, a cellular phone link, an RF link or other communications channels.

Computer programs (also called computer control logic) are stored in main memory 1508 and/or secondary memory 1510. Computer programs may also be received via communications interface 1524. Such computer programs, when executed, enable computer system 1500 to implement embodiments of the present invention as discussed herein, such as the computing modules. In particular, the computer programs, when executed, enable processor 1504 to implement the methods of embodiments of the present invention, including the methods implemented by the computing modules. Accordingly, such computer programs represent controllers of the computer system 1500. Where the invention is implemented using software, the software may be stored in a computer program product and loaded into computer system 1500 using removable storage drive 1514, interface 1520, hard drive 1512 or communications interface 1524.

### VI. Conclusion

Set forth above are example systems, methods, and computer-program products for remotely classifying materials based on complex permittivity features. While various embodiments of the present invention have been described above, it should be understood that they have been presented by way of example only, and not limitation. It will be apparent to persons skilled in the relevant art that various changes in form and detail can be made therein without departing from the spirit and scope of the invention.

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way. Thus, the breadth and scope of the present invention should not be limited by any of the above-described exemplary embodiments, but should be defined only in accordance with the following claims and their equivalents.

1. A system for remotely detecting contraband material in an arbitrary enclosure, comprising:
a first electrode configured to generate an electric field;
a second electrode configured to:
sense interaction of the electric field with a container and a plurality of materials within the container, wherein the first and second electrodes are arranged orthogonal to a plane of the container; and
a computing module configured to:
derive from the sensing a plurality of permittivity parameters corresponding to the materials within the container;
present the plurality of permittivity parameters using a multi-dimensional vector in a multi-dimensional space;
project the multi-dimensional vector into a lower-dimensional space;
organize the projected vector, corresponding to the plurality of permittivity parameters, into a plurality of clusters in the lower dimensional space;
compare the plurality of clusters against a plurality of known signatures for a plurality of previously identified contraband materials; and
detect and identify at least one of the materials in the container as being a contraband material based on the comparison, wherein a location of a first cluster of the plurality of clusters corresponds to a known signature of the contraband material.

2. The system of claim 1, wherein the first electrode and the second electrode are configured as opposing-plate electrodes.

3. The system of claim 1, wherein the first electrode and the second electrode are configured as a fringing-field array.

4. The system of claim 3, further comprising:
a guide structure configured to support the first and second electrodes.

5. The system of claim 4, further comprising:
a motor configured to cause the first and second electrodes to move along the guide structure.

6. The system of claim 5, further comprising:
a support structure configured to support a sample and positioned substantially perpendicular to the guide structure, wherein the sample comprises the contraband material.

7. The system of claim 6, wherein the motor is further configured to cause the sample to move along the support structure.

8. The system of claim 1, wherein the first electrode is configured to generate an electric field having a single frequency.

9. The system of claim 1, wherein the first electrode is configured to generate an electric field having a plurality of different frequencies.

10. (canceled)

11. The system of claim 1, wherein:
the first electrode is one electrode of a first plurality of electrodes that are each configured to generate an electric field; and
the second electrode is one electrode of a second plurality of electrodes that are each configured to sense the interaction of the electric field with the container and any materials in the container and provide a signal corresponding thereto.

12. The system of claim 11, wherein one or more electrodes in the first plurality of electrodes is selected at a given time to generate the electric field.

13. The system of claim 11, wherein one or more electrodes in the second plurality of electrodes is selected at a given time to sense the interaction of the electric field with the container and any materials in the container and provides a signal corresponding thereto.

14. (canceled)

15. A method for remotely detecting contraband material, comprising:
generating, by a first electrode, an electric field;
sensing, by a second electrode, interaction of the electric field with a container containing a plurality of materials within the container, wherein the first and second electrodes are arranged orthogonal to a plane of the container;
deriving, by a computing module, from the sensing a plurality of permittivity parameters corresponding to the materials within the container;
presenting, by the computing module, the plurality of permittivity parameters using a multi-dimensional vector in a multi-dimensional space;
projecting, by the computing module, the multi-dimensional vector into a lower-dimensional space;
organizing, by the computing module, the projected vector, corresponding to the plurality of permittivity parameters, into a plurality of clusters in the lower dimensional space;
comparing, by the computing module, the plurality of clusters against a plurality of known signatures for a plurality of previously identified contraband materials; and
detecting and identifying, by the computing module, at least one of the materials in the container as being a contraband material based on the comparison, wherein a location of a first cluster of the plurality of clusters corresponds to a known signature of the contraband material.

16. The method of claim 15, wherein:
generating an electric field comprises generating an electric field using a first electrode; and
sensing interaction comprises sensing interaction using a second electrode to sense the interaction of the electric field with the container and any materials in the container, wherein the first and second electrodes are configured as opposing-plate electrodes.

17. The method of claim 15, wherein:
generating an electric field comprises generating an electric field using a first electrode; and
sensing interaction comprises sensing interaction using a second electrode to sense the interaction of the electric field with the container and any materials in the container, wherein the first and second electrodes are configured as a fringing-field array.

18. The method of claim 17, further comprising:
supporting the first and second electrodes on a guide structure.

19. The method of claim 18, further comprising:
moving the first and second electrodes along the guide structure.

20. The method of claim 19, further comprising:
supporting the material on a support structure, wherein the support structure is positioned substantially perpendicular to the guide structure.

21. The method of claim 20, further comprising:
moving the material along the support structure.

22. The method of claim 15, wherein the generating an electric field comprises generating an electric field having a single frequency.

23. The method of claim 15, wherein the generating an electric field comprises generating an electric field having a plurality of different frequencies.

24. (canceled)

25. The method of claim 15, wherein:
generating an electric field is carried out by a first plurality of electrodes and sensing interaction of the electric field is carried out by a second plurality of electrodes.

26. The method of claim 25, wherein the first plurality of electrodes and the second plurality of electrodes are configured as opposing-plate electrodes.

27. The method of claim 25, wherein the first plurality of electrodes and the second plurality of electrodes are configured as a fringing-field array.

28. The method of claim 25, further comprising:
sequentially selecting one or more of the first plurality of electrodes to generate the electric field.

29. The method of claim 25, further comprising:
sequentially selecting one or more of the second plurality of electrodes to sense the interaction of the electric field with the container and any materials in the container.

30. A tangible non-transitory computer-readable medium having stored thereon computer-executable instructions that, if executed by a device, cause the device to perform a method for detecting contraband material in a container, the method comprising:
deriving a plurality of permittivity parameters corresponding to a plurality of materials within the container from an electrode configured to sense interaction of an electric field with the container and the materials in the container, wherein the electrode is arranged orthogonal to a plane of the container;
presenting the plurality of permittivity parameters using a multi-dimensional vector in a multi-dimensional space;
projecting the multi-dimensional vector into a lower-dimensional space;
organizing the projected vector, corresponding to the plurality of permittivity parameters, into a plurality of clusters in the lower dimensional space;
comparing the plurality of clusters against a plurality of known signatures for a plurality of previously identified contraband materials; and
detecting and identifying at least one materials in the container as being a contraband material based on the comparison, wherein a location of a first cluster of the plurality of clusters corresponds to a known signature of the contraband material.

31. (canceled)

32. The system of claim 1, wherein the computing module is further configured to:
find a subspace of the multi-dimensional space, in which the permittivity parameters have a largest variance, as the lower-dimensional space.

33. The system of claim 32, wherein to find the subspace, the computing module is further configured to use linear discriminant analysis (LDA).

34. The system of claim 32, wherein the computing module is further configured to:
organize the permittivity parameters of a known material into a second cluster in the subspace.

35. The system of claim 1, wherein the computing module is further configured to:
use a distance to measure similarity between a probability distribution of the plurality of permittivity parameters of different materials.

36. The system of claim 35, wherein the distance is a bhattacharyya distance.

37. The system of claim 1, wherein the container is an automobile.

38. The system of claim 32, wherein dimensions of the subspace represent a cross-section of the multi-dimensional space.

39. A system for remotely identifying a plurality of contraband materials in an arbitrary enclosure, comprising:
a first electrode configured to generate an electric field;
a second electrode configured to:
sense interaction of the electric field with a container and the plurality of contraband materials within the container, wherein the first and second electrodes are arranged orthogonal to a plane of the container; and
a computing module configured to:
derive from the sensing a plurality of permittivity parameters corresponding to the plurality of contraband materials;
present the plurality of permittivity parameters using a multi-dimensional vector in a multi-dimensional space;
project the multi-dimensional vector into a lower-dimensional space;
compare a plurality of clusters corresponding to the plurality of contraband material in the container against a plurality of known signatures for a plurality of previously identified contraband materials; and
detect each of the plurality of contraband materials by clustering the projected vector in the lower dimensional space;
identify at least one material in the container as being contraband material based on the comparison, wherein a location of a first cluster of the plurality of clusters corresponds to a known signature of the contraband material.

40. The system of claim 1, wherein the plurality of known signatures are derived based on at least separating electrical parameters of the previously identified contraband material into different clusters.

41. The system of claim 1, wherein the permittivity parameters are derived based on at least a distortion of the electric field by the materials of the container, and wherein the second electrode measures a voltage caused by the electric field.
